(19) United States
(12) Patent Application Publication
(10) Pub. No.: US 2021/0145494 A1
(43) Pub. Date: May 20, 2021

(54) BURR HOLE COVER

(71) Applicant: OsteoMed LLC, Addison, TX (US)

(72) Inventors: Benjamin Carl Casey, Dallas, TX (US); Justin L. Rowland, Atlanta, GA (US); Peter Nakaji, Phoenix, AZ (US)

(21) Appl. No.: 16/951,973

(22) Filed: Nov. 18, 2020

Related U.S. Application Data
(60) Provisional application No. 62/937,180, filed on Nov. 18, 2019.

(51) Int. Cl. A61B 17/80

(52) U.S. Cl. CPC A61B 17/8085; A61B 2017/568; A61B 17/808

(57) ABSTRACT

A burr hole cover comprising a central cover, a plurality of arms, and a plurality of struts is described. At least some of the plurality of arms extend from the central cover and define apertures designed to receive surgical screws. Each of the plurality of struts are designed to connect a first portion of the burr hole cover to a second portion of the burr hole cover. The plurality of struts are configured to be selectively removed to provide for adjustable rigidity of one or more portions of the burr hole cover.

### CROSS-REFERENCE TO RELATED APPLICATION

The present application claims priority to U.S. Provisional Application No. 62/937,180 filed Nov. 18, 2019 and entitled “BURR HOLE COVER,” the disclosure of which is incorporated by reference herein in its entirety.

### TECHNICAL FIELD

The present disclosure generally relates to burr hole covers that may be used by surgeons during a neurosurgical procedure and methods for manufacturing such covers.

### BACKGROUND

Craniotomy is a surgical procedure that is generally performed to treat for neurosurgical conditions and diseases. A craniotomy, in some cases, involves forming one or more burr holes. Burr holes may be small holes, e.g., 8-10 mm in diameter, created in the skull through to the level of the dura. Burr holes provide a surgeon access to the brain and allow the surgeon to perform a desired neurosurgical procedure. Illustrative neurosurgical procedures include surgically implanting deep brain stimulators for the treatment of Parkinson's disease, epilepsy, and cerebellar tremor, passing drainage catheters that allow for cerebrospinal fluid drainage, or evacuation of chronic buildup of blood within the skull. Forming burr holes often leads to skull defects, which further results in small but undesirable scalp depressions. This defect is usually unacceptable to the patient from a cosmetic perspective. Moreover, the scalp depression may aggravate over time with the resolution of wound swelling in the early stage and the atrophy of soft tissue in the late stage, and it causes a cosmetic complex to the patients especially during hairdressing or combing.

Numerous types of burr hole covers made from different types of materials have been tested to treat the skull defect. However, each one of them has issues. For instance, burr hole covers made up of autologous bone, muscle, or fat tissue are highly biocompatible, but are linked with donor site complications, are time consuming, and are difficult to apply. Whereas, polymethyl methacrylate (PMMA)-based covers can be applied, but it is time consuming and has a thermal reaction which may be toxic to surrounding tissues. Mineral graft such as hydroxyapatite (HA) is not toxic to the tissues and has osteoconductive properties, but it is often too brittle and its resorption easily takes place when cerebrospinal fluid or water is present. Polyethylene is biocompatible, available in various sizes, and easy and quick to apply. However, its poor cost-effectiveness can be a disadvantage. Titanium-based burr hole covers have shown promise; however, the currently used titanium-based burr hole covers may be undesirably more pliable than what is necessary and lack required rigidity/structural support. In addition, titanium-based burr hole covers require surgical screws to secure the cover with the underlying skull bone. However, the head of the screws may protrude out after the cover is secured. The protruding screw heads may appear as undesirable crests on the skin, which may be an issue for the patient from a cosmetic perspective. Furthermore, the screw head may erode the tissue underneath, which can displace the whole closure apparatus from its desired position.

### SUMMARY

The present application describes burr hole cover devices with enhanced structural features for improving burr hole cover outcomes. The instant application describes using a burr hole cover having a central cover portion to cover the underlying burr hole. The central cover portion has a plurality of arms extending therefrom (e.g., extending away from the central cover portion). The burr hole cover further includes a plurality of struts, which may be configured to provide additional rigidity to the burr hole cover. The plurality of struts may be designed to connect two portions of the burr hole cover. For example, in embodiments, at least one of the plurality of struts may be designed to connect a first arm of the plurality of arms with a second arm of the plurality of arms. The burr hole cover is also designed to be fixated to a patient's skull. For example, in one embodiment each of the plurality of arms defines an aperture that is designed to receive a fixation device, such as a surgical screw, which fixates the burr hole cover to the skull. In embodiments, the burr hole cover also includes an affixation plate/portion that defines the aperture. For example, one of the plurality of arms may include an affixation plate/portion positioned at a distal end of the arm and defining the aperture therein. In embodiments, the affixation plate may be designed to prevent the fixation device from protruding. For example, the affixation plate may be designed to be placed in a perforation on the skull so as to receive a fixation device (e.g., surgical screw) and prevent the head of the fixation device from protruding.

In some embodiments, the burr hole cover may be patient specific. In one embodiment, at least the plate portion of the burr hole cover is individually custom designed for every patient according to the contour of the patient's skull (which is created from various medical imaging techniques (e.g., CT scans, MRI scans, and the like)). In some embodiments, the burr hole cover is designed to be generically designed to be used on multiple patients, such as using multiple different sizes to provide a semi-custom fit. The present application also describes various embodiments of methods for manufacturing these burr hole covers. The burr hole covers, in some embodiments, may be manufactured using 3D printing techniques, and the like.

The foregoing has outlined rather broadly the features and technical advantages of the embodiments in order that the detailed description of the embodiments that follows may be better understood. Additional features and advantages of the embodiments disclosed in this application will be described hereinafter which form the subject of the claims of the application. It should be appreciated by those skilled in the art that the conception and specific embodiment disclosed may be readily utilized as a basis for modifying or designing other structures for carrying out the same purposes of the present application. It should also be realized by those skilled in the art that such equivalent constructions do not depart from the spirit and scope of the embodiments in this application as set forth in the appended claims. The novel features which are believed to be characteristic of the embodiments, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding, reference is now made to the following descriptions taken in conjunction with the accompanying drawings, in which:

FIG. 1(a) depicts an illustrative burr hole cover, in accordance with embodiments of the present disclosure;

FIG. 1(b) depicts a three dimensional (3D) rendered image of a portion of skull;

FIG. 2 depicts another illustrative burr hole cover, in accordance with embodiments of the present disclosure;

FIG. 3 depicts yet another illustrative burr hole cover, in accordance with embodiments of the present disclosure; and

FIG. 4 depicts an illustrative method that may be performed by a surgeon, in accordance with embodiments of the present disclosure.

### DETAILED DESCRIPTION

For the sake of illustration and clarity, this disclosure describes medical devices, such as burr hole covers, having various different designs. However, it should be appreciated that the disclosure is not intended to be limited to the examples and designs of burr hole covers, but is to be accorded the widest scope consistent with the principles and novel features of the burr hole covers, including burr hole covers having one or more struts that connect two portions of the burr hole cover to improve the structural rigidity of the burr hole cover. Such struts may be designed to be detachable (e.g. by snipping and the like) to provide for flexibility in the device if desired. The description ahead is provided to enable any person skilled in the art to make or use the disclosure. Various modifications to the disclosure will be readily apparent to those skilled in the art, and the generic principles of the use and manufacturing of the burr hole covers defined herein may be applied to other variations as well.

The description below illustrates various designs of burr hole covers. Each of the design includes the following features: a central cover portion designed to cover a burr hole or couple with a bone flap; one or more arms extending radially outward from the central cover portion; apertures defined in each of the arms to accept one or more fixation devices; and one or more struts connecting different portions of the burr hole cover. Burr hole covers described below, in one embodiment, include titanium and have a thickness of about 0.5 mm. However, the thickness of the burr hole covers is not limited to 0.5 mm. In some embodiments, the thickness of the burr hole covers may be uniform throughout, meaning that the thickness of the central cover portion may be substantially similar to the thickness of the arms. In other embodiments, the thickness of the central cover portion may be different (e.g. thicker) than the thickness of the arms.

The struts connecting different portions of the burr hole cover improve the structural rigidity of the burr hole covers as the struts resist pressure in the direction of its length and make the arms relatively less pliable. Thus, the structural rigidity of burr hole covers including one or more struts is better than the structural rigidity of burr hole covers that do not include the one or more struts. In some embodiments, one or more of the struts may be removed (e.g., by snapping or breaking) so as to provide a surgeon with a flexibility to make adjustments to the aperture placement (or placement of affixation plate that defines the aperture) when fixating the burr hole covers to the underlying skull. The flexibility or pliability of the arms (and, in corollary, the pliability of the burr hole cover) may be increased by a surgeon during surgery by breaking or snapping one or more of the struts.

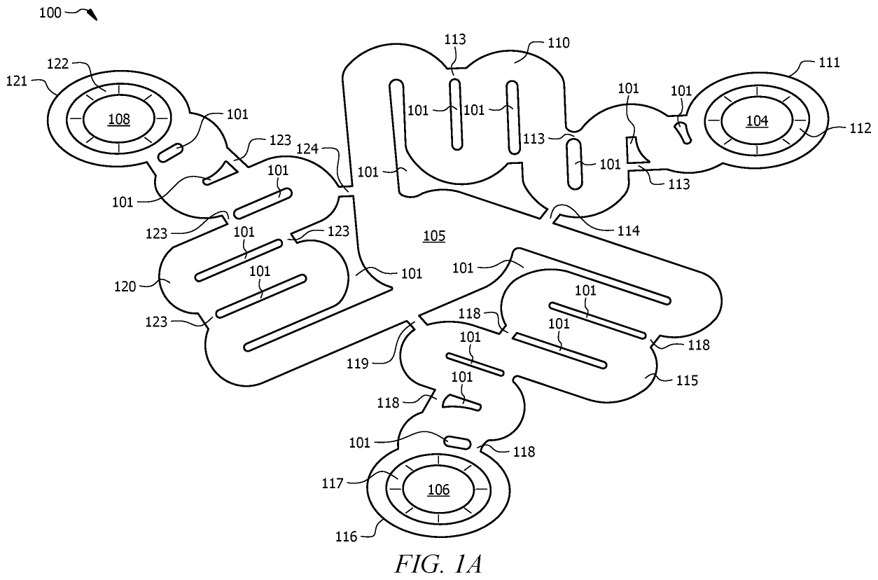

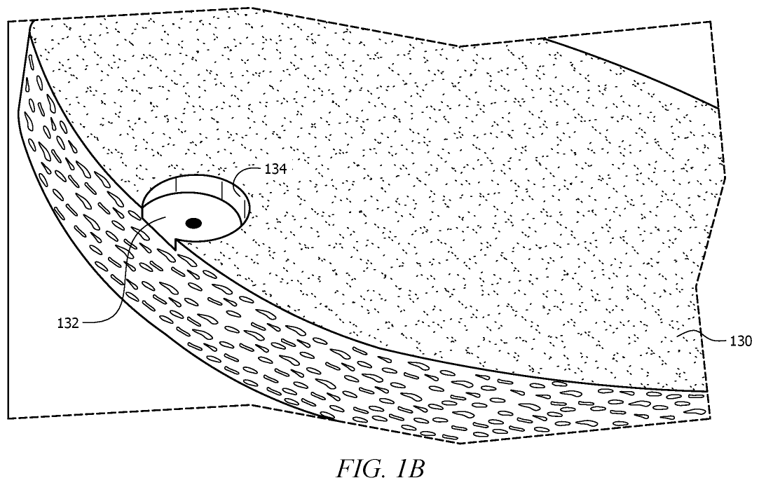

Referring to FIG. 1(a), an illustrative burr hole cover 100 having arms 110, 115, and 120 with a serpentine design is shown. Burr hole cover 100 includes center cover 105, which is shown to be a solid cover without holes or perforations in it. In embodiments, the center cover 105 may define features (e.g., apertures) on it. The features, in some instances, may promote bone growth. Burr hole cover 100 may further include arms 110, 115, and 120 that radially extend outward from center cover 105. Arms 110, 115, and 120 may also include affixation plates/portions 104, 106, and 108, respectively which may be positioned at distal ends of their respective arms. In embodiments, the affixation plates 104, 106, and 108 may be continuously formed as part of burr hole cover 100 or alternatively formed as separate portions. Affixation plates 104, 106, and 108 may define apertures (e.g., holes) that are designed to receive surgical fixating devices (e.g., surgical screws) and fixate burr hole cover 100 to a patient's skull. For example, apertures defined in affixation plates 104, 106, and 108 may be designed to accommodate screws to fasten burr hole cover 100 into the underlying skull bone.

Arms 110, 115, and 120 of burr hole cover 100 may include perforations 101 that allow access to the brain, e.g., allow for shunt or drain access. In some embodiments, each of the arms 110, 115, and 120 includes one or more struts that connect one portion of a particular arm with another portion of the particular arm. For example, arm 110 includes strut 113 which connects different curvy, serpentine portions of arm 110 to each other. In a similar manner, arm 115 includes strut 118 which connects different curvy, serpentine portions of arm 115 to each other, and arm 120 includes strut 123 which connects different curvy portions of arm 120 to each other. Struts 113, 118, and 123 improve structural rigidity of burr hole cover 100 over previous design.

In some embodiments, burr hole cover 100 includes struts, such as strut 124, that connect one arm to another arm. For example, burr hole cover 100 includes strut 124 that connects arm 110 with arm 120. Burr hole cover 100 further includes strut 114 that connects arm 110 to arm 115 and strut 119 that connects arm 115 with arm 120. Struts 114, 119, and 124 may also be designed and positioned improve the structural rigidity of burr hole cover 100. In some embodiments, struts 113, 114, 118, 119, 123, and 124 may be designed to be snapped (or broken/snipped, and the like) as desired by the surgeon during surgery. For example, while covering a burr hole using burr hole cover 100, a surgeon may snap one or more struts (e.g., struts connecting a same arm or struts connecting different arms) to make adjustments to the affixation plate (or the aperture) placement when fixating the burr hole covers to the underlying skull. In embodiments, the surgeon, after snapping one or more struts, may stretch the curvy, serpentine design of the arm to place the aperture at a desired position. The serpentine design of arms 110, 115, and 120 and the snapping feature of the struts allow for covering a large surface area over the skull. In embodiments, one or more struts may connect one arm to the central cover.

Each affixation plate (e.g., affixation plates 104, 106, and 108) has a top surface and an inner surface. The inner surface is on the other side of the top surface and touches the underlying bone (e.g., the skull). For instance, affixation plate 104 has top surface 111 and inner surface 112; affixation plate 106 has top surface 116 and inner surface 117; and affixation plate 108 has top surface 121 and inner surface 122. Each of the affixation plates may include an aperture (or an opening) that is designed to receive a fixation device, such as a surgical screw, to fixate burr hole cover 100 to the skull. Each affixation plate may also be designed to be placed in a perforation on the skull so as to prevent a fixation device (e.g., a surgical screw) to protrude. The contour of each of the perforations in the skull can be imagined to be mirror images of the outer contour of their respective affixation plates. In other words, the contour of each of the perforations in the skull may follow the outer contour of their respective affixation plates. In one embodiment, the outer contour of each of the affixation plates is tapered and thus has a wider diameter at the top surface than the inner surface. The perforation that immerses the foregoing tapered contour also, thus, has a wider diameter at the top and a narrow surface at the bottom.

In another embodiment, the outer contour of each of the affixation plates is vertical and thus has substantially equal diameter at both the top and the inner surfaces. For example, referring to FIG. 1(b), a three dimensional (3D) rendered image 129 of a portion of skull 130 is shown. Image 129 shows a perforation 132 having a contour 134 that is a mirror image of the outer contour of an affixation plate, for instance affixation plate 104. In this case, the contour of the affixation plate 104 allows the affixation plate 104 to immerse in the perforation 132 when is placed there by the surgeon. The surgical screw, when drilled into the aperture (defined in the affixation plate which is immersed into the perforation 132), also positions itself in the affixation plate at the same height as the top surface of the affixation plate, thus preventing the screw head from protruding. In embodiments, the different affixation plates may be sized differently. For example, the geometric characteristics (e.g., tapered length, top diameter, bottom diameter, etc.) of one affixation plate (e.g., affixation plate 104) may be different (e.g., longer) with respect to another affixation plate (e.g., affixation plate 106). In embodiments, the geometric characteristics may depend on the thickness of skull bone at which the affixation plate is positioned. In embodiments where the geometric characteristics of the affixation plates are different from one another, the surgical screws are sized accordingly.

In operation, after performing the craniotomy procedure, the surgeon may experience some issues (e.g., space restrictions) while cover the underlying burr hole using burr hole cover 100. In order to overcome that, the surgeon may choose to break or snap one or more of the struts to increase the flexibility of one or more of the arms to secure the burr hole cover at a desired location on the skull. For example, while securing burr hole cover 100, the surgeon may snap or break strut 124 and/or one or more of struts 123 to make arm 120 more pliable and flexible (and alter the original shape of the burr hole cover 100) to secure burr hole cover 100 at a desired location on the skull. In some cases, the surgeon may choose to elongate one or more of arms 110, 115, and 120 to secure burr hole cover 100. In that case, the surgeon may snap or break one or more of the struts related to the elongated arm(s). In summary, the flexibility or pliability of the arms (and, in corollary, the pliability of the burr hole cover) may be increased by a surgeon during surgery by breaking or snapping one or more of the struts as needed. Stated another way, struts are configured to be selectively removed to provide for adjustable rigidity of one or more portions of the burr hole cover. The one or more portions may include arms of the burr hole cover.

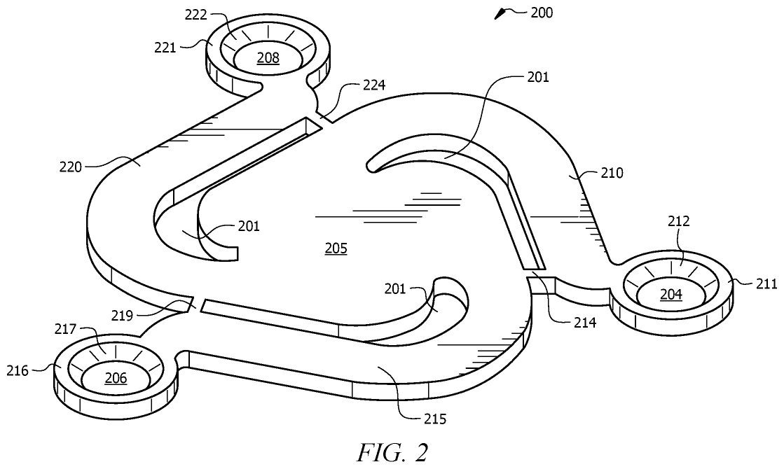

Referring now to FIG. 2, an illustrative burr hole cover 200 with another design is shown. Burr hole cover 200 includes center cover 205, which may have a solid cover design (e.g., without holes or perforations in it). In embodiments, the center cover 205 may have features (e.g., apertures) defined on it. Burr hole cover 200 may also include arms 210, 215, and 220 that radially extend outward from center cover 205. Arms 210, 215, and 220, similar to arms of burr hole cover 100, may include affixation plates 204, 206, and 208 (respectively) that may be positioned at the distal ends of their respective arms. In embodiments, the affixation plates may define apertures that fixate burr hole cover 100 to a patient's skull. For example, arms 210, 215, and 220 may define apertures that are designed to accommodate screws to fasten burr hole cover 200 into the underlying skull bone (e.g., in the manner described above).

Arms 210, 215, and 220 of burr hole cover 200 include perforations 201 in between the arms and the center cover which may allow brain access or to allow for future bone growth. In comparison to the arms of burr hole cover 100, arms 210, 215, and 220 do not include perforations defined in them. Burr hole cover 200 includes struts that connect one arm with another to improve the rigidity of the burr hole cover 200. In contrast to the arms of burr hole 100, the arms of burr hole cover 200 do not include one or more struts that connect one portion of a particular arm with another portion of the same arm. For example, burr hole cover 200 includes strut 224 that connects arm 210 with arm 220. Similarly, strut 214 connects arm 210 to arm 215, and strut 219 connects arm 215 with arm 220. In some embodiments, struts 214, 219, and 224 may be designed to be snapped or broken as desired by the surgeon during surgery (e.g., in the manner described above). The number of struts that may be present between two arms is not limited to what is shown in FIG. 2. The number may vary in other designs. In some embodiments, burr hole cover 200 includes struts (not shown) that connect one arm with center cover 205 to improve the rigidity of the burr hole cover 200.

Similar to the affixation plates of burr hole cover 100, each of the affixation plates 204, 206, and 208 have a top surface and an inner surface. For instance, affixation plate 204 has top surface 211 and inner surface 212; affixation plate 206 has top surface 216 and inner surface 217; and affixation plate 208 has top surface 221 and inner surface 222. Similar to the corresponding description in FIG. 1(A), each of the affixation plates 204, 206, and 208 may include an aperture (or an opening) that is designed to receive a fixation device, such as a surgical screw, to fixate burr hole cover 200 to the skull. Each affixation plate 204, 206, and 208 may be also designed to be placed in a perforation on the skull so as to prevent a fixation device (e.g., a screw) to protrude. Similar to the description above, the contour of each of the perforations in the skull can be imagined to be mirror images of the outer contour of their respective affixation plates. In one embodiment, the outer contour of each of the affixation plates is tapered and thus has a wider diameter at the top surface than the inner surface. In another embodiment, the outer contour of each of the affixation plates is vertical and thus has substantially equal diameter at both the top and the inner surfaces.

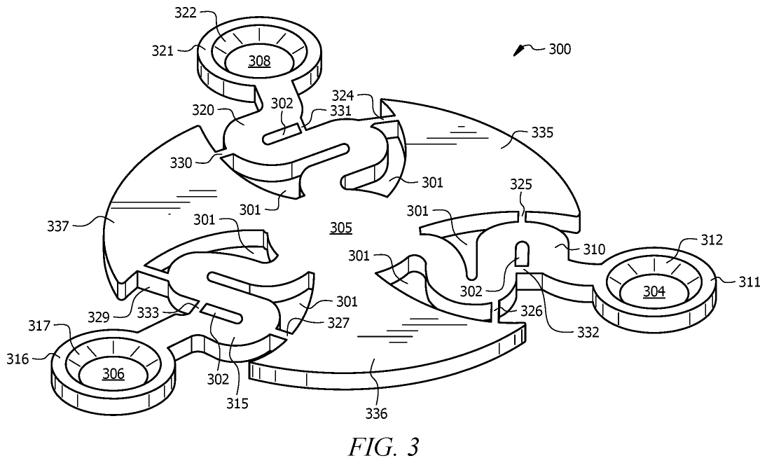

Referring now to FIG. 3, an illustrative burr hole cover 300 is shown. Burr hole cover 300 is comprised of center cover 305 having additional surface plates 335, 336, and 337, which in combination to center cover 305 cover a burr hole. In embodiments, center cover 305 and the additional surface plates are solid covers without holes or perforations in them. However, other embodiments may include center cover 305 with holes or perforations for various purposes (e.g., to promote bone growth). Burr hole cover 300 includes arms 310, 315, and 320 radially extending outward from center cover 305 and positioned between the surface plates (e.g., arm 310 is positioned between surface plates 335 and 336). In embodiments, burr hole cover may define perforations 302 that may allow access to the brain (e.g., allow for shunt or drain access). For example, arms 310, 315, and 320 may define perforations 302 in them. The arms may also include affixation plates 304, 306, and 308 that further define apertures that fixate burr hole cover 300 to a patient's skull. For example, arms 310, 315, and 320 may include affixation plates 304, 306, and 308, respectively define apertures designed to receive and accommodate surgical fixation devices (e.g., surgical screws) to fasten burr hole cover 300 into the underlying skull bone.

In embodiments, burr hole cover 300 may include additional perforations (e.g., perforations 301). For example, perforations 301 may be defined in between the arms and the additional surface plates of center cover 305 to allow brain access and/or to allow for future bone growth. Similar to the designs described above, burr hole cover 300 include struts that connect different portions of the burr hole cover 300. For example, burr hole cover 300 include struts (e.g., strut 333) that connect one portion of a particular arm (e.g., arm 315) with another portion of the particular arm. Burr hole cover 300 may further includes struts that connect an arm to a different portion of the burr hole cover 300, e.g., central plate (or a portion thereof) to improve the rigidity of the burr hole cover 300. For example, burr hole cover 300 includes: struts 325 and 326 that connect arm 310 with additional surface plates 335 and 336, respectively; struts 327 and 329 that connect arm 315 with additional surface plates 336 and 337, respectively; and struts 324 and 330 that connect arm 320 with additional surface plates 335 and 337, respectively. Similar to the description made above with respect to burr hole covers 100 and 200, struts of burr hole cover 300 may be designed to be snapped (or broken) as desired by the surgeon during surgery (e.g., provide flexibility/pliability).

Similar to the affixation plates of burr hole cover 100 and 200, each of the affixation plates 304, 306, and 308 has a top surface and an inner surface. For instance, affixation plate 304 has top surface 311 and inner surface 312; affixation plate 306 has top surface 316 and inner surface 317; and affixation plate 308 has top surface 321 and inner surface 322. Each affixation plate shown in FIG. 3 is also designed to be placed in a perforation on the skull so as to prevent a fixation device (e.g., a screw) to protrude. Similar to the description of the affixation plate above, the contour of each of the perforations in the skull can be imagined to be mirror images of the outer contour of their respective apertures. In one embodiment, the outer contour of each of the affixation plates is tapered and thus has a wider diameter at the top surface than the inner surface. In another embodiment, the outer contour of each of the affixation plates in vertical and thus has substantially equal diameter at both the top and the inner surfaces.

The choice of using a particular design of a burr hole cover may depend on the surgeon's preference. For example, the surgeon may desire the apertures to be relatively closer to the central cover portion. In that case, the surgeon may choose to use the design described in FIG. 2 over the design described in FIG. 1(a). In some embodiments, a type of design used by a surgeon may depend on the type of surgery, anatomy of the patient, size of the burr hole, etc.

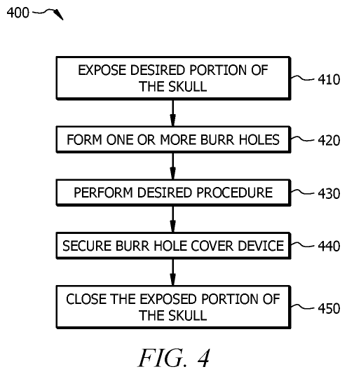

Referring now to FIG. 4, an illustrative method 400 that may be performed by a surgeon is shown. In particular, method 400 illustrates the use of a burr hole cover over a burr hole during a craniotomy procedure. Method 400, in one embodiment, begins with block 410 that includes exposing a desired portion of the skull. This may be performed by first incising the scalp above the desired portion of the skull with a scalpel, then exposing the desired portion using a retractor, and then removing the periosteal layer over the desired portion. Method 400 may then move to block 420 that includes forming one or more burr holes. The number of burr holes formed may depend on the type of surgery. For illustration's sake, it is assumed that the surgeon forms one burr hole. The one burr hole may be formed by using a high-speed drill which drills a small hole (e.g., 7-9 mm in diameter). Method 400 may then move to block 430 that include performing the desired procedure, which may include one of the many intracranial procedures that may be performed by the surgeon. After performing the procedure, method 400 may proceed to block 440 that includes securing the burr hole formed in block 420. Securing the burr hole, in one embodiment, includes taking one of the burr hole covers described in FIGS. 1(a), 2, and 3 and placing the central portion of the burr hole cover over the burr hole and placing the arms at their corresponding locations per the design of the selected burr hole cover. Securing the burr hole device may include creating perforations in the skull, where the shape of the perforations is determined by the outer contour of the apertures defined in the arms. Perforations for the apertures may be created such that the apertures immerse in the perforations. Securing the burr hole device further includes drilling surgical screws in the apertures immersed inside the perforations. During the securing/placement of the cover, the surgeon may break or snap one or more struts to stretch/bend/expand one or more arms for placement on the skull. This may include extending one or more arms to one or more perforations placed in the skull. Method 400, in one embodiment, concludes in block 450 that includes closing the exposed portion of the skull.

In some embodiments, the burr hole cover may be patient specific. In one embodiment, at least the plate portion of the burr hole cover is custom designed individually for every patient according to the contour of patient's skull (which is created from various medical imaging techniques (e.g., CT scans, MRI scans, and the like)). In some embodiments, the burr hole cover may not be patient specific. In that case, the burr hole covers are not designed for a specific patient, but are designed in accordance with generic human anatomical features. Thus, the same design can be used to produce multiple burr hole covers, which can further be used as burr hole covers of different patients. In some embodiments, these non-patient-specific guides may be designed based on age, gender, or generic physical makeup of the human anatomical structure. The burr hole covers, in some embodiments, may be manufactured using 3D printing techniques, and the like.

In some embodiments, a method of manufacture of the burr hole covers includes receiving a patient's data, e.g., one or more electronic images of the site to be operated. As noted above, the electronic images of the sites may be from, without limitation, a CT image, a spiral CT image, an MRI image, an ultrasound scan, digital tomosynthesis, or optical coherence tomography. The received patient data, in one embodiment, may then be utilized to generate 3D bone models of the skull. The 3D bone model, in one embodiment, is generated using a computer system configured to receive the images and/or other details and generate the bone model (e.g., of the skull) using a software system installed in the computer system. The method of manufacture may also include 3D fabricating the burr hole cover to be used in the surgery. As noted above, the burr hole covers are manufactured using additive technology or freeform fabrication. In this method of manufacture, the burr hole covers are formed through successive fusion of chosen parts of powder layers applied to a worktable. In some embodiments, PA 12 (also known as Nylon 12) or titanium is used as the powder. The burr hole covers formed using titanium have high tensile strength, impact strength, and are able to flex without fracture. In other embodiments, other types of material may be used. In summary, once the patient-specific information is ascertained, rapid prototyping or other manufacturing techniques may be used to adapt the burr hole cover to the patient's particular biological structure. Burr hole covers that are not designed for a specific patient can also be fabricated using similar method of manufacture.

The method of manufacture (for both patient specific or generic burr hole cover) may include accessing a computer-readable medium having stored thereon three-dimensional (3D) images of a burr hole cover and fabricating burr hole cover based on their 3D images. The method of fabrication may include fabricating a central cover (e.g., cover 105) to be positioned over a burr hole created in a skull. The method further include fabricating a first arm (e.g., arm 115) and a second arm (e.g., arm 120) extending radially outward from the central portion and including an aperture (e.g., apertures 106 and 108) designed to receive a surgical fixating device. The method yet further include fabricating a breakable strut connecting a first portion (e.g., first arm, second arm, or the central cover) of the burr hole cover and a second portion (e.g., first arm, second arm, central cover, or the additional surface plates) of the burr hole cover. It is noted that the above-described fabrication steps may be part of a single 3D print or molding technique.

Although embodiments of the present application and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps. Moreover, the scope of the present application is not intended to be limited to the particular embodiments of the process, machine, manufacture, composition of matter, means, methods and steps described in the specification.

1. A burr hole cover, comprising:
a central cover;
a plurality of arms extending from the central cover, wherein at least some of the plurality of arms define apertures designed to receive surgical screws; and
a plurality of struts, wherein each of the plurality of struts are designed to connect a first portion of the burr hole cover to a second portion of the burr hole cover, and wherein the plurality of struts are configured to be selectively removed to provide for adjustable rigidity of one or more portions of the burr hole cover.

2. The burr hole cover of claim 1, wherein a first arm of the plurality of arms comprises the first and second portion of the burr hole cover.

3. The burr hole cover of claim 1, wherein a first arm of the plurality of arms comprises the first portion and a second arm of the plurality of arms comprises the second portion.

4. The burr hole cover of claim 1, wherein a first arm of the plurality of arms comprises the first portion and the central cover comprises the second portion.

5. The burr hole cover of claim 1, wherein a first arm of the plurality of arms comprises the first portion and one of an additional surface of the central cover comprises the second portion.

6. The burr hole cover of claim 1, wherein each of the at least some of the plurality of arms include an affixation plate having a top surface and an inner surface.

7. The burr hole cover of claim 6, wherein the affixation plate is designed to prevent surgical screws from protruding above a top surface defined by the affixation plate.

8. The burr hole cover of claim 1, wherein the at least some of the apertures are designed to be immersed in perforations in a skull.

9. A medical device having one or more removable struts connecting portions of the medical device, the medical device comprising:
a central cover designed to be positioned over a burr hole created in a skull;
a first arm and a second arm, the first and second arms extending radially outward from the central cover;
a first affixation plate positioned at a distal end of the first arm and a second affixation plate positioned at a distal end of the second arm, wherein at least one of the first and second affixation plates includes an aperture designed to receive a surgical fixating device; and
one or more removable struts, wherein each of the one or more removable struts is designed to connect a first portion of the medical device and a second portion of the medical device.

10. The medical device of claim 9, wherein the first and second arms define perforations within or between them to allow for brain access.

11. The medical device of claim 9, wherein an outer contour of the first affixation plate follows a contour of a perforation created for the first affixation plate.

12. The medical device of claim 11, wherein the outer contour of the first affixation plate allows the aperture to be immersed in the perforation.

13. The medical device of claim 11, wherein the outer contour of the first affixation plate is tapered.

14. The medical device of claim 9, wherein the first portion comprises the central cover and the second portion comprises the first arm.

15. The medical device of claim 9, wherein one of the one or more removable struts connects the first portion of the first arm with the second portion of the first arm.

16. The medical device of claim 9, wherein the first portion comprises the first arm and the second portion comprises the second arm.

17. The medical device of claim 9 further comprising one or more additional surface plates extending away from the central cover and configured to be positioned over the burr hole.

18. The medical device of claim 17, wherein the first portion comprises one of the one or more additional surface plates and the second portion comprises the first arm.

19. The medical device of claim 9, wherein the first and second affixation plates are designed to prevent surgical fixating devices from protruding above a top surface defined by at least one of the first and second affixation plates.

20. A method of manufacturing a burr hole cover, comprising:
accessing a computer-readable medium having stored thereon three-dimensional (3D) images of a burr hole cover;
fabricating burr hole cover based on one or more of the 3D images, wherein the fabricating the burr hole cover includes:
fabricating a central cover to be positioned over a burr hole created in a skull;
fabricating a first arm extending radially outward from the central portion and including an aperture designed to receive a surgical fixating device; and
fabricating a removable strut connecting a first portion of the burr hole cover and a second portion of the burr hole cover.
